(19) United States
(12) Patent Application Publication
(10) Pub. No.: US 2023/0102127 A1
(43) Pub. Date: Mar. 30, 2023

(54) SYSTEMS AND METHODS FOR IDENTIFYING SAMPLES OF INTEREST BY COMPARING ALIGNED TIME-SERIES MEASUREMENTS

(71) Applicant: BASE SE, Ludwigshafen (DE)

(72) Inventors: Michael LISZKA, San Diego, CA (US); Mark WALL, San Diego, CA (US)

(21) Appl. No.: 17/797,677

(22) PCT Filed: Feb. 2, 2021
(86) PCT No.: PCT/US2021/016208

§ 371 Date: Aug. 4, 2022

Related U.S. Application Data
(60) Provisional application No. 62/969,915, filed on Feb. 4, 2020.

(51) Int. Cl. G01N 27/447; G01N 33/68

(52) U.S. Cl. CPC G01N 27/44717; G01N 33/6803; G01N 27/44782

(57) ABSTRACT

Example embodiments provide systems and methods for identifying samples of interests by comparing aligned time-series measurements, For example, the techniques described herein may be used to, among other applications, perform data capture, processing, and analysis of high-throughput capillary electrophoresis data for protein identification. Other applications include analysis of DNA and RNA samples, and/or polysaccharides. Time-series measurements may be collected from an analysis instrument and automatically aligned based, e.g., on peaks in the data. The aligned peaks of test samples and control samples may be programmatically compared to identify samples of interest; in some embodiments, the data peaks may be permitted to float within a predefined window so as to improve the quality of the comparison and provide more meaningful results. The system may generate an output including an identifier of a sample of interest, images of spectral peaks, and/or tables of time-series measurements.

### BACKGROUND

Identifying new materials such as proteins, deoxyribonucleic acid (DNA), ribonucleic acid (RNA), polysaccharides, etc. can lead to breakthroughs in chemistry, medicine, biology, and other fields. It is accordingly desirable to identify new variations of these materials quickly and efficiently. Analysis tools have been developed to capture data that can be used to rapidly analyze samples of these materials. However, once these analyses arc complete, an expert must generally review the data in order to determine which samples are most likely to yield interesting formulations.

This task is complicated by the fact that cross-sample data may not be standardized or directly comparable due to (e.g.) variations in the way the analysis is performed from sample-to-sample, in the instruments used to analyze different samples, in the samples themselves, etc. Moreover, the data must be curated so that it is useful internally within an organization and externally, if made available outside the organization. As a result, the identification of new materials can be time-consuming, expensive, and resource-intensive.

### SUMMARY

Exemplary embodiments provide methods, mediums, and systems for programmatically identifying samples of interest by comparing time-series measurements of test samples against control samples.

According to one embodiment, a system may interface with an analysis instrument configured to analyze a collection of samples. The samples may include a test sample and a control sample.

The system may receive results of an analysis of the collection of samples, where the analysis includes time-series measurements for the collection of samples. In some embodiments, the samples include a protein, DNA, RNA, and/or polysaccharides. The sample of interest may include a material not present in the control sample and may be identified based on an electrophoresis analysis.

In some embodiments, the time-series measurements include spectral absorbance measurements, phosphorescence measurements, fluorescence measurements, voltage measurements, and measurements of other physical quantities including energy, force, torque, light, or position. Any of these measurements may be converted to an electrical signal and read by the system.

The system may align the time-series measurements of the collection of samples. In some embodiments, aligning the time-series measurements may include identifying one or more first peaks in the time-series measurement of the test sample, identifying one or more second peaks in the time-series measurement of the control sample, and allowing the first peaks and the second peaks to float relative to each other within a predefined tolerance window.

The system may programmatically identify a sample of interest by comparing the aligned time-series measurements of a test sample and a control sample. The sample of interest may be identified because the control sample does not include a component of interest and test sample does include a component of interest. In some embodiments, programmatically identifying the sample of interest involves subtracting a first time-series measurement of the control sample from a second time-series measurement of the test sample. In some embodiments, a distance (such as a Euclidean distance) between the time-series measurement of the control sample and the time-series measurement of the test sample may be computed. The control sample to be compared to a given test sample may be selected based on identifying that the control sample has the smallest computed distance from the test sample from among to the plurality of control samples.

The system may generate an output including an identifier for the identified sample of interest. In some embodiments, the output may include a table of spectra peaks, which the system may automatically submit to a database of experimental results identified by the system. In some embodiments, the output may include an image of spectra peaks; in some cases, a user may select peaks of interest (e.g., from the table or an image of multiple peaks corresponding to a sample or samples), and the image of the spectra peaks may include peaks corresponding to those selected in the data.

These and other embodiments will now be described with reference to the attached drawings.

### BRIEF DESCRIPTION OF THE DRAWINGS

FIG. 1 depicts an environment suitable for use with exemplary embodiments.

FIGS. 2A-2C depict examples in which time-series measurements are aligned.

FIGS. 2D-2E depict side-by-side examples in which peaks from a control sample is subtracted from the test sample (FIG. 2D) and in which the size of the peaks of the test sample arc divided by the size of the peaks from the control sample (FIG. 2E).

FIG. 3A depicts an example of an input data structure suitable for use with exemplary embodiments.

FIG. 3B depicts an example of an output data structure suitable for use with exemplary embodiments.

FIG. 4 depicts an input/output specification for an exemplary embodiment.

FIG. 5 is a block diagram depicting logic according to an exemplary embodiment.

FIG. 6 is a flowchart illustrating an exemplary procedure suitable for practicing exemplary embodiments.

FIG. 7 depicts an exemplary computing system suitable for use with exemplary embodiments.

FIG. 8 depicts an exemplary network environment suitable for use with exemplary embodiments.

### DETAILED DESCRIPTION OF EXAMPLE EMBODIMENTS

Exemplary embodiments provide techniques for identifying samples of interests by comparing aligned time-series measurements, For example, the techniques described herein may be used to, among other applications, perform data capture, processing, and analysis of high-throughput capillary electrophoresis data for protein identification. Other applications include analysis of DNA and RNA samples, and/or polysaccharides. Time-series measurements may be collected from an analysis instrument and automatically aligned based, e.g., on peaks in the data, The aligned peaks of test samples and control samples may be programmatically compared to identify samples of interest; in some embodiments, the data peaks may be permitted to float within a predefined window so as to improve the quality of the comparison and provide more meaningful results. The system may generate an output including an identifier of a sample of interest, images of time-series peaks, and/or tables of time-series measurements.

Using the system described herein. materials of interest can be identified orders of magnitude more quickly than using traditional methods, which saves cost, time, and resources, and allows for more efficient data reuse. For example, in one trial run nearly 400 samples were analyzed in less than an hour—a nearly tenfold efficiency increase. A given plate of electrophoresis samples can be analyzed in some situations in less than thirty seconds, Moreover, because the system eliminates a significant resource bottleneck, more samples can be analyzed compared to conventional techniques.

The following description of embodiments provides non-limiting representative examples referencing numerals to particularly describe features and teachings of different aspects of the invention. The embodiments described should be recognized as capable of implementation separately, or in combination, with other embodiments from the description of the embodiments. The description of embodiments should facilitate understanding of the invention to such an extent that other implementations, not specifically covered but within the knowledge of a person of skill in the art having read the description of embodiments, would be understood to be consistent with an application of the invention.

It is noted that, although exemplary embodiments are described in connection with particular examples (electrophoresis analysis of proteins, a standalone alignment and identification system, etc.), the present invention is not limited to these examples.

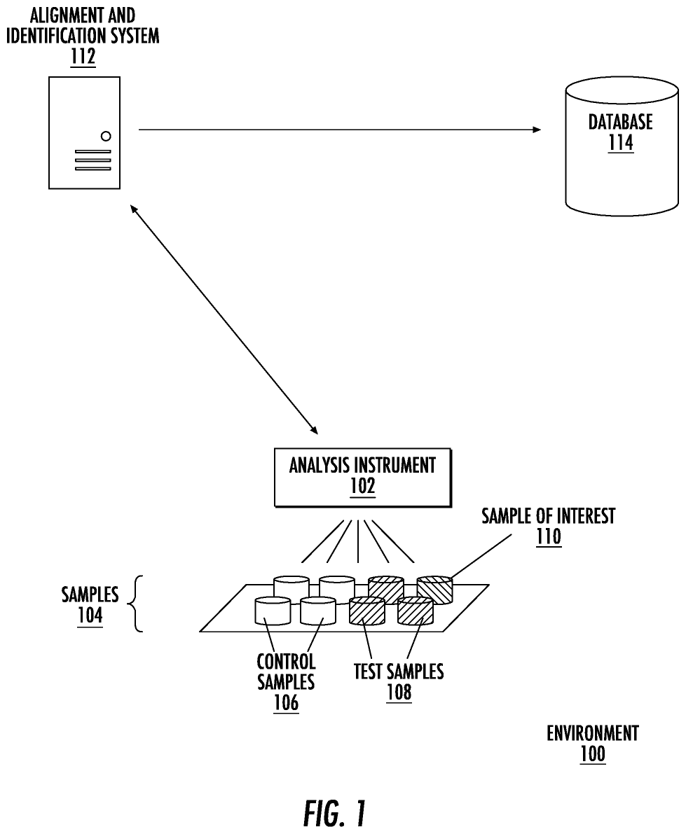

FIG. 1 illustrates an environment 100 according to an example embodiment.

The environment 100 includes an analysis instrument 102, and alignment and identification system 112, and a database 114.

The analysis instrument 102 may interact with a set of samples 104 in order to gather time-series data. For example, the analysis instrument may be a high-throughput (HTP) capillary electrophoresis instrument configured to analyze samples 104 including proteins. The samples might alternatively (or in addition) include deoxyribonucleic acid (DNA), ribonucleic acid (RNA), and/or polysaccharides. The different types of materials may be segregated into different samples 104, or may be present together in a sample 104. In the case of HTP capillary electrophoresis, the time-series measurements may include spectral absorbance measurements, in which case the measurements may represent (e.g.) molecular masses observed in the sample over time. In other embodiments, the analysis instrument may be configured to perform other types of time-series analyses, such as by gathering fluorescence data, phosphorescence data, or voltage data.

The samples 104 may include control samples 106 and test samples 108. The control samples 106 may include set of samples that include known proteins (or other materials) which are identified as not being of interest. The test samples 108 may or may not include other materials that are of interest; the alignment and identification (A&I) system 112 may test the compares the test samples 108 against the control samples 106 to identify whether any materials of interest are present. To this end, the analysis instrument 102 may collect time-series data from the samples 104, and then provide the time-series data to the A&I system 112 for alignment and identification.

For example, if a test sample 108 includes a target protein having a given molecular mass, the time-series data will include an extra peak (as compared to one of the control samples 106) at a location corresponding to the molecular mass of the target protein. The test samples 108 may be compared against the control samples 106 in order to identify one or more samples of interest 110 from among the test samples 108. The sample of interest 110 may be a sample that has one or more characteristics not present in the control samples 106. For instance, the sample of interest 110 may be identified because it includes one or more proteins (or other materials of interest) that arc not present in the control samples.

Thus, the control sample 106 serves as a background to be contrasted with selected test samples 108. By subtracting out this background, samples of interest 110 that include target materials (which may or may not be known a priori) can be identified for re-analysis, further analysis, or some other use. If a sufficient amount of material is left over after subtracting out the background (e.g., more than a predetermined threshold amount), then the system may identify the selected test sample 108 as a sample of interest 110. The predetermined threshold amount may be adjusted in order to edit the sensitivity of the comparison.

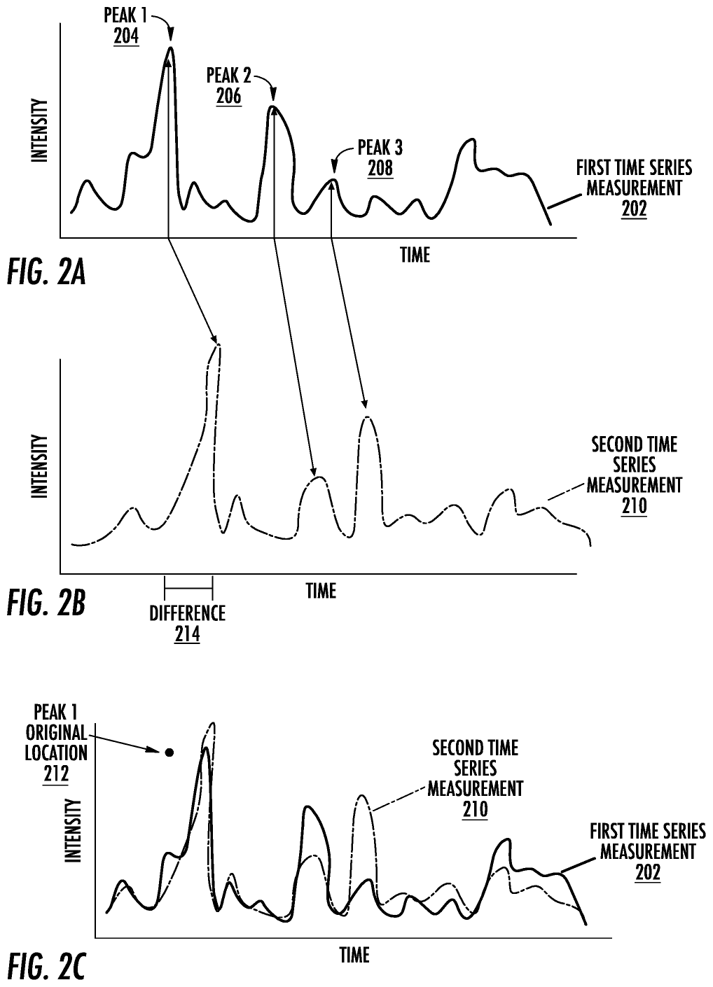

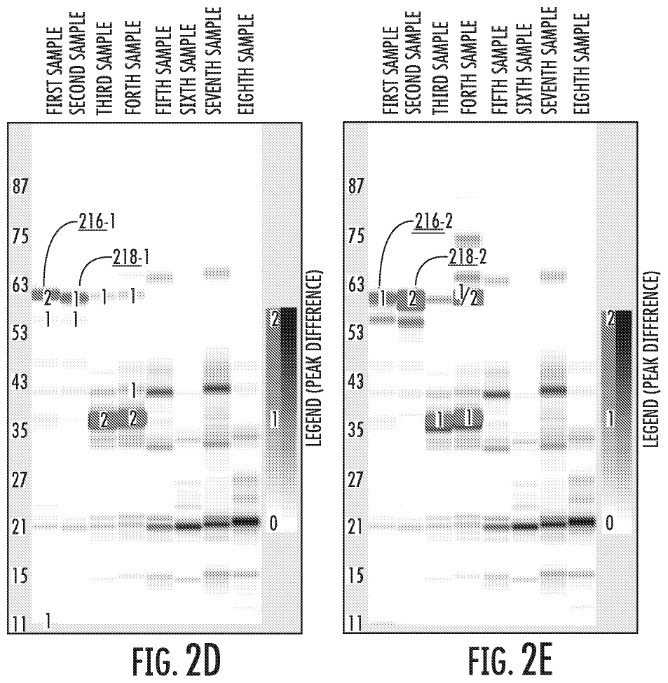

Moreover, as described in more detail below, the time-series measurements of the control samples 106 and the test samples 108 may need to be aligned (e.g., by the A&I system 112) before a comparison can be made. This alignment can be performed based on one or more peaks present in a control sample 106 and a test sample 108. An exemplary procedure for performing this alignment is described below with respect to FIGS. 2A-2C.

FIG. 2A depicts a graph of a first set of time-series measurements 202 as obtained (e.g.) from a control sample 106. Peaks 204, 206, 208 may be identified in the measurements 202. The peaks 204, 206, 208 may be, for example, local maximums in the graph of the measurements 202. In some cases, only a limited number (e.g., a predetermined amount) of peaks may be identified (e.g., the N highest peaks), or some of the peaks may be limited from consideration if they arc too close (c.g., within a predetermined distance) to another (higher) peak.

FIG. 2B depicts a second graph of time-series measurements 210, corresponding (e.g.) to a test sample under consideration. As can be seen by comparing FIG. 2A to FIG. 2B, the second time series measurements 210 includes peaks that generally correspond to the location of the peaks in the first time-series measurements 202, although they are shifted to the left by a difference 214. FIG. 2C shows the first time series measurements 202 overlaid on the second time-series measurements 210, after the first time series measurements 202 are shifted by the difference 214. Accordingly, for example, the original location 212 of peak 1 204 is moved to the right until it aligns with the corresponding first peak in the second time series measurements 210. Once this alignment procedure is completed, the difference between the first time series measurements 202 and the second time series measurements 210 may be computed by subtracting one from the other.

This alignment procedure may be performed because of variations from analysis to analysis, which can arise for a number of reasons. Even the same sample, analyzed with the same procedure at two different times, may yield results that are shifted, compressed, or expanded. Thus, the alignment procedure could shift one or both of the graphs of the time- series measurements by a certain distance, or could expand or compress the time-series measurements, or could perform a combination of expansion/compression and shifting. Generally, the entire set of data will be modified in the same way, but in some circumstances, it may be appropriate to apply different transformations to different portions of the data.

When transforming the data, it may be important to identify a maximum allowable transformation amount, or a window in which the data is permitted to float. If the data is allowed to be shifted by too great an amount, then the sets of measurements become distorted, and comparison of the two sets is meaningless: any one point in a reference measurement set can be compared to any point in a comparison set. On the other hand, if the window is set too small, it may not be possible to develop a reasonable comparison, and samples of interest may be missed because it is not possible to accurately subtract out the background data.

In some cases, multiple control samples 106 may be available, and a particular control sample may be selected for comparison to a given test sample 108. In order to find the optimal control sample 106 to match against a selected test sample 108, the time-series data from each candidate control sample 106 may be aligned to the selected test sample 108, and the aligned data may be compared. For example, a distance between the candidate control sample 106 and the selected test sample 108 may be calculated at different time points. The distance may be a Euclidean distance. The control sample 106 that minimizes the distance with the selected test sample 108 may be selected as the control sample for comparison to the selected test sample 108.

In some embodiments, the distance may be calculated without shifting, expanding, or compressing the data. In such a static comparison, no attempt is made at peak alignment. In other embodiments, referred to as dynamic time weighting, the peaks are allowed to float within a window as described above. In one exemplary embodiment, the size of the window (for data measured in terms of molecular mass) may be set to a few kilodaltons.

As noted above, after the peaks are aligned, differences the aligned peaks may be calculated. The differences may be calculated by (for example) subtracting the peaks of the control sample from the peaks of the test sample, as shown in the spectral measurements of FIG. 2D. In this example, any residual peak value left over after subtracting out the control sample may signify that materials of interest are present in the test sample. These differences are highlighted in the spectra using (e.g.) a darker color and/or numerical values. For example, in the first sample, a first peak 216-1 has been identified as being different from a corresponding peak in a control sample. The medium-dark color and modest numerical value (“1”) assigned to the first peak 216-1 indicate that this peak is moderately different from the corresponding peak in the control sample to which it is being compared. On the other hand, a first peak 218-1 in a second sample has also been identified as being different from a corresponding peak in a control sample, but exhibits a greater difference (relatively more so than the first peak 216-1 of the first control sample). Accordingly, the color assigned to the first peak 218-1 of the second control sample is darker than the first peak 216-1 of the first control sample, and the numerical value assigned to the first peak 218-1 of the second control sample is higher (“2”).

Other mathematical operations may also be performed to identify the differences between the samples in order to perform comparable analyses. For example, FIG. 2E depicts an example where the size of the peaks in the test samples have been divided by the size of corresponding peaks in the control samples. When the peaks arc very close, this value should approach “1.” As the size of the peaks diverge, the number will grow (or shrink, if the peak of the control sample is larger than the peak of the test sample). In this example, the dominant test peaks are apparent in both FIGS. 2D and 2E, while minor peaks appear more significantly in FIG. 2E. This division analysis could be extended to identify decreases in the test peaks relative to the control peaks.

Other embodiments may perform other types of mathematical operations in order to identify samples of interest; they arc not limited to subtraction or multiplication, but may use any suitable mathematical operation. Some types of mathematical analysis may be more or less sensitive to differences in the peak sizes, and hence may be more suitable for different applications or contexts.

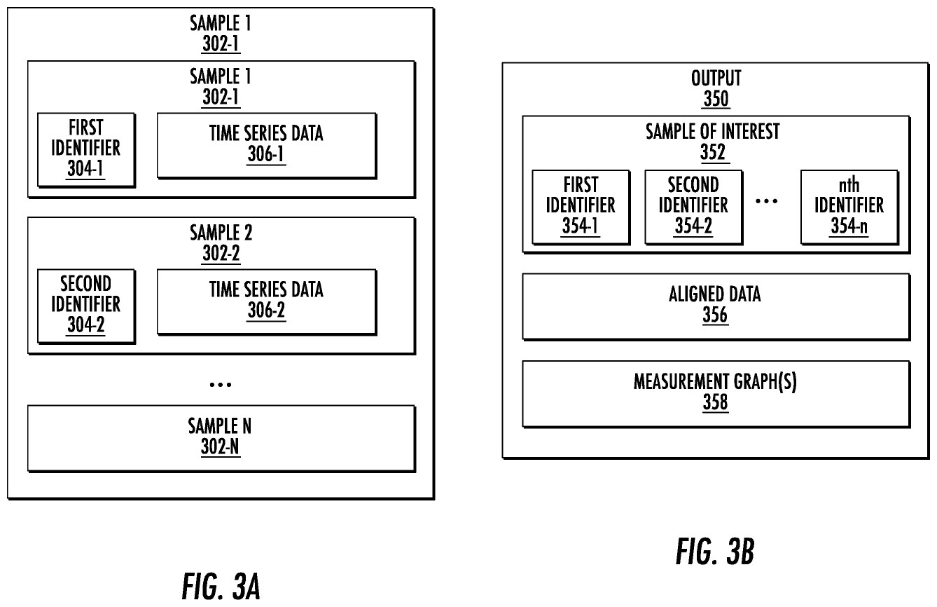

In order to perform the above-described analysis, the A&I system 112 may receive an input 300 from the analysis instrument 102; an exemplary input 300 data structure suitable for use with exemplary embodiments is depicted in FIG. 3A.

The input 300 may be divided into a number AT of samples 302-i (where i=1−N). Each portion of the input 300 data structure associated with a given sample 302-i may include an identifier 304-i for the ith sample, and time-series data 306-i derived from an analysis of the ith sample by the analysis instrument.

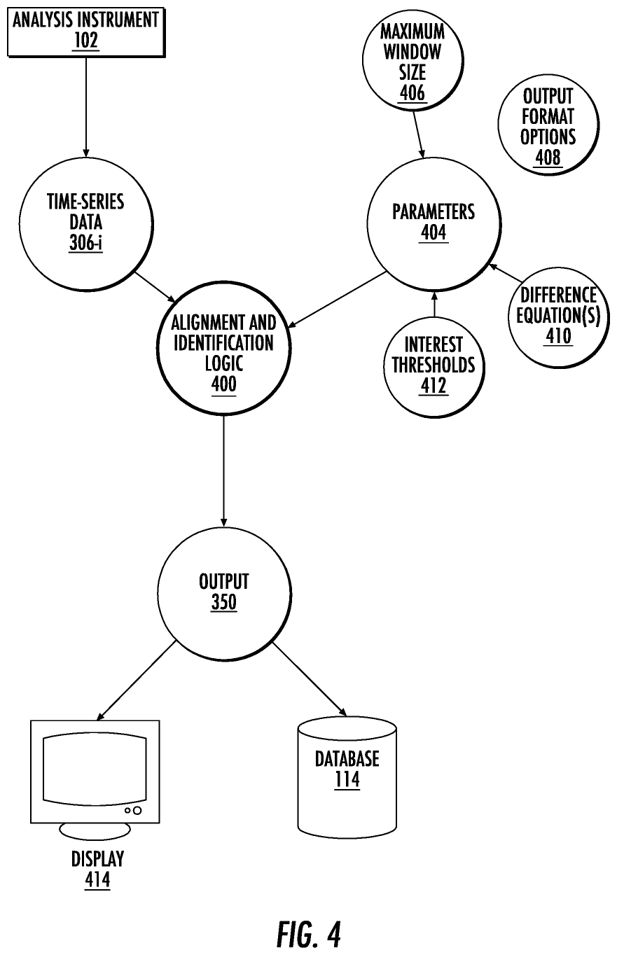

Using the information from the input 300 data structure (and, optionally, additional parameters and settings as shown in FIG. 4), the A&I system 112 may generate an output 350. FIG. 3B depicts an example of an output data structure 350 suitable for use with exemplary embodiments. The A&I system 112 may generate the output data structure 350 and may store it locally, display all or portions of the output data structure 350, and/or transmit the output data structure 350so that it can be stored in a remote database.

The output 350 may include identifiers 354-i for any samples that have been identified by the A&I system 112 as being of interest (the identifiers 354-i may correspond to the identifiers 304-i identified in the input 300 structure).

The output 350 may further include the aligned data 356 corresponding to all of the time-series data 306-i, or the subset of time-series data 306-i corresponding to the samples of interest 352. The data may be in any suitable format, such as a comma-separated value (CSV) list, an array, a linked list, a matrix, a table, a custom data structure, etc.

The output 350 may further include measurement graphs 358 generated from the time-series data 306-i and/or the aligned data 356. The measurement graphs 358 and/or the aligned data 356 may be displayed on a display device configured to display the data. Using the display, a user may perform actions such as filtering the data, flagging some samples of interest 352 as not being of interest, adding samples that had not originally been flagged as of interest, comparing aligned control and test samples, comparing different test samples, etc.

If A&I system 112 is configured to display information from the output 350 data structure, then the A&I system 112 may display either or both of the tabular or graphical data, which may include a number of peaks. The peaks may be highlighted in the tabular and/or graphical data. In one embodiment, the graphical/tabular data from a given test sample may be overlaid or displayed side-by-side with the data from the control sample against which it was compared. Differences between the test sample and the control sample may be visually distinguished (e.g., by highlighting the aligned peaks, coloring the peaks in a color different from the default color for the rest of the data, adding shading in a peak that extends above another peak to which it is aligned, and/or adding in numerical values representing a difference between two aligned peaks, among other possibilities).

In some embodiments, some aligned peaks that exhibit differences may be programmatically categorized as not significant, and some may be categorized as significant. For instance, if a peak extends above another peak to which it is aligned by less than a predetermine threshold amount, the peak may be discounted as not significant. In some embodiments, peaks flagged as not significant may be excluded from consideration when subtracting out the background data to determine if the target sample is a sample of interest.

As shown in the exemplary input/output specification of FIG. 4, the time-series data 306-i may be provided from the analysis instrument 102 to alignment and identification logic 400, which may be provided (e.g.) on the A&I system 112.

The A&I system 112 may also make use of (locally or remotely-stored) parameters 404. The parameters 404 may include, for example, a maximum size 406 of the window through which the peaks are allowed to float; in some embodiments, the maximum amount of compression or expansion may alternatively or additionally be provided as part of the parameters 404. The parameters 404 may further include output format options 408, which describe which options are included in the output 350 data structure, how those options are displayed, etc. The parameters 404 may further include one or more equations 410 or techniques for computing the difference between two sets of time-series measurements (e.g., in order to identify a control suitable for comparison to a given test sample, and/or to subtract out the background represented by the control from the test sample in order to identify whether the test sample includes materials of interest. In some embodiments, the difference must meet a certain minimum threshold before the sample is identified as being of interest; the parameters 404 may therefore also include one or more interest thresholds 412 that define how much of a difference is required before a sample is flagged as being of interest.

After processing the time-series data 306-i in view of the parameters 404, the A&I logic 400 may generate an output 350. The output 350 may be provided to a display 414 to display the data in tabular and/or graphical format, and/or may be uploaded to a database 114 of experimental results.

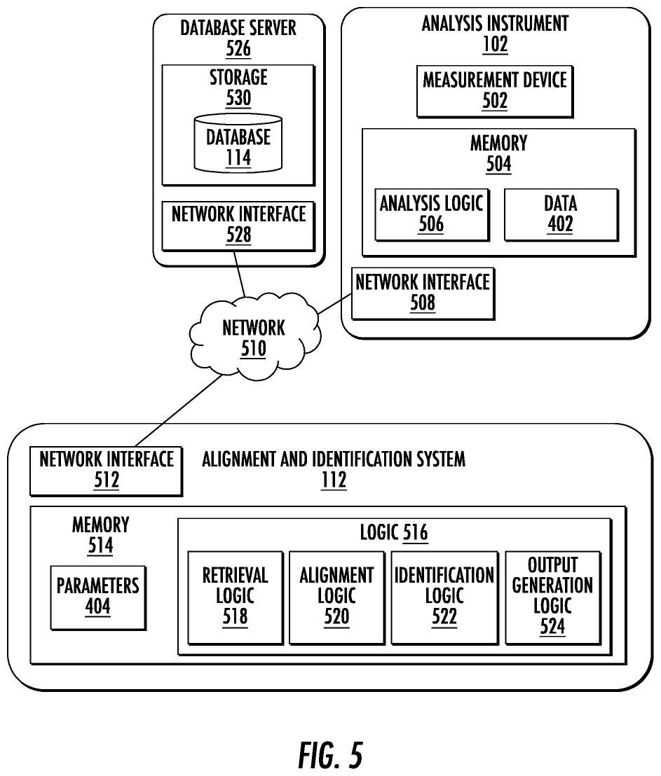

FIG. 5 is a block diagram depicting logic deployed on the devices of the environment 100 according to an exemplary embodiment.

The analysis instrument 102 includes a measurement device 502 suitable for analyzing a collection of samples. For instance, the measurement device 502 may be an HTP capillary electrophoresis device, a device suitable for analyzing fluorescence or phosphorescence, or an electrical testing device suitable for reading voltage measurements from the samples.

The analysis instrument 102 may include a memory 504, which may be any suitable non-transitory computer-readable medium (e.g., RAM, ROM, an HDD, an SSD, flash memory, etc.). The memory 504 may store, as one or more instructions executable by a processor on the analysis instrument 102 (not shown), analysis logic 506 for performing an analysis of the samples using the measurement device 502.

The memory 504 may further store data 402, which may be time-series measurements generated by the analysis logic 506. The data 402 may be provided to the A&I system 112 over a network 510 via corresponding hardware network interfaces 508, 512.

The A&I system 112 may also include a memory 514 (e.g., a non-transitory computer-readable medium), which may store the parameters 404 used to align the time-series measurements and/or identify samples of interest. The memory 514 may store, as one or more instructions executable by a processor on the analysis instrument 102 (not shown), logic 516 for aligning control and test samples, and identifying test samples of interest.

The output may be provided, in one embodiment, via the network interface 512 to a corresponding hardware network interface 528 on a database server 526. The database server 526 may host a storage device (a non-transitory computer-readable medium) configured to add the output from the A&I system 112 to the database 114.

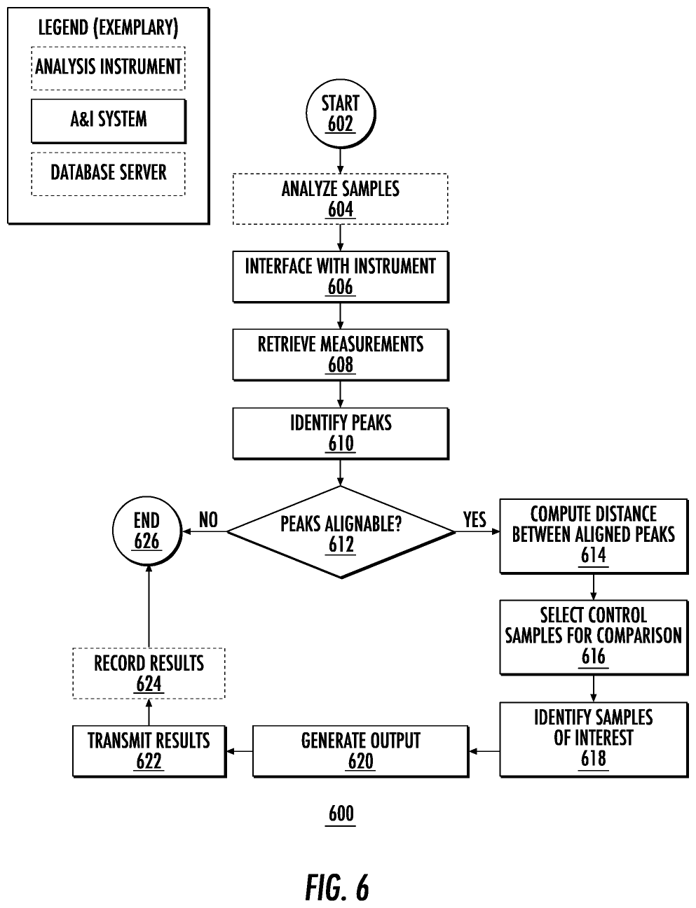

The logic 506 and 516 is described in more detail in connection with the flowchart 600 of FIG. 6. For example, the analysis logic 506 may perform the actions described at block 604; the retrieval logic 518 may perform the actions described at block 608; the alignment logic 520 may perform the actions described at blocks 610-616; the identification logic 522 may perform the actions described at block 618; and the output generation logic 524 may perform the actions described at block 620.

With reference to FIG. 6, processing may start at block 602.

At block 604, the analysis instrument may analyze a group of samples according to a design or configuration of the analysis instrument. For example, the analysis instrument may perform an electrophoresis measurement, a fluorescence measurement, a phosphorescence measurement, a voltage measurement, or any other suitable measurement. The measurements performed by the analysis instrument may be collected and organized as time-series data and stored in a memory of the analysis instrument.

At block 606, the A&I system may interface with the analysis instrument, such as by establishing a connection to the analysis instrument over a network. In some embodiments, the A&I system may be connected directly (in a wired and/or wireless manner) to the analysis instrument. In other embodiments, the A&I system may be integrated with the analysis instrument, and thus the two devices may already be connected.

At block 608, the A&I system may retrieve the measurements stored at the analysis instrument via the interface established at block 606. The analysis instrument may provide the data to the A&I system using the input format shown in FIG. 3A.

At block 610, the A&I system may identify one or more peaks in the data retrieved at block 608. The peaks may be, for instance, local maximums in the data. As noted above, in some embodiments the system may refrain from identifying a value as a peak if it is too close to another, higher peak (e.g., within a predetermined threshold distance and/or less than a predetermined difference in height as compared to the higher peak).

After the peaks are identified in the data, at block 612, the system determines if the in the peaks in the control data and the target data are alignable. For example, the system may retrieve a window size representing the maximum amount by which the peaks of the control and/or target data are allowed to float with respect to each other. In some embodiments, the time-series data may be compressed and/or stretched in order to align the control data to the test data.

If the determination at block 612 is “no” (i.e., there is no suitable control sample that corresponds to a given test sample), then an analysis of the test sample may not be possible; processing may proceed to block 626 and end. Alternatively, if more data remains to be analyzed, the system may move on to the next test sample for analysis and processing may return to block 610 with a new sample.

If the determination at block 612 is “yes” (i.e., that at least one control sample exists that can be aligned to the test sample), then processing may proceed to block 614. Optionally, if only one control sample aligns to the test sample, processing may skip ahead to block 616 and the one control sample may be selected as the sample for comparison.

Next, the A&I system may identify a closest control sample to which a particular test sample may be aligned. To that end, at block 614, the A&I system may compute a distance (e.g., a Euclidean distance) between each candidate control sample and the test sample in question, and may select the control sample having the least distance from the test sample as the sample for comparison (block 616).

At block 618, the system may identify whether any of the test samples are samples of interest. In this block, the control sample may be treated as background measurements, which are subtracted from the corresponding aligned measurements of the aligned test sample. Any values remaining after the background data is subtracted may correspond to material of interest. Samples having a sufficient amount of remaining value (e.g., above a predetermined threshold) after subtracting out the background data may be flagged as samples of interest. In some embodiments, these samples may be preliminary flagged, subject to review by a user. Those samples of interest that are selected (or not eliminated) may be added to data in an output data structure (block 620).

At block 622, the output data structure may be transmitted to a suitable database and/or a display. Accordingly, the A&I system may consult the parameters stored at the A&I system to determine output options for the data. If the data is to be displayed on a display, the A&I system may format the data appropriately and transmit the data to the display. If the data is to be stored in a database (e.g., a database of experimental results), the A&I system may identify a suitable database at block 622 and transmit the results to a corresponding database server/service for storage in the database. At block 624, the database may receive the data and store it. Processing may then proceed to block 626, and terminate.

Although FIGS. 1-6 depict specific components in a particular configuration, it is contemplated that other configurations may also be used. For example, any or all of the analysis instrument 102, the A&I system 112, and the database 114 may be integrated in a single device, or aspects of these systems and instruments may be separated into distinct devices. Various logic modules may be split into multiple modules or combined into a single module, and may be deployed on a single device (which may or may not be the precise device noted above), or split between multiple devices.

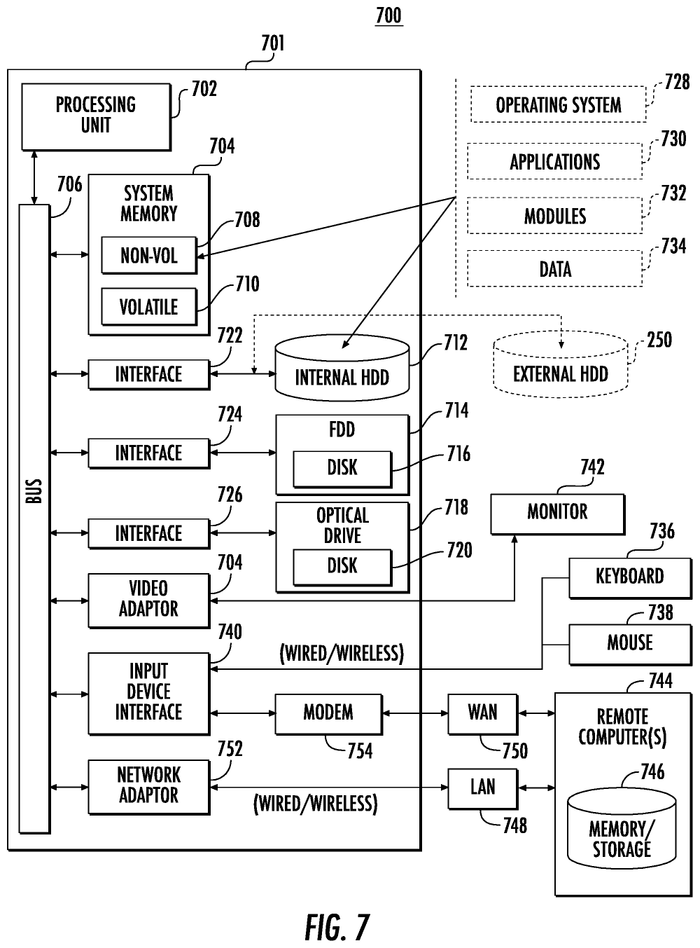

The above-described methods may be embodied as instructions on a computer readable medium or as part of a computing architecture. FIG. 7 illustrates an embodiment of an exemplary computing architecture 700 suitable for implementing various embodiments as previously described. In one embodiment, the computing architecture 700 may comprise or be implemented as part of an electronic device, such as a computer 701. The embodiments arc not limited in this context.

As used in this application, the terms “system” and “component” are intended to refer to a computer-related entity, either hardware, a combination of hardware and software, software, or software in execution, examples of which arc provided by the exemplary computing architecture 700. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 700 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 700.

As shown in FIG. 7, the computing architecture 700 comprises a processing unit 702, a system memory 704 and a system bus 706. The processing unit 702 can be any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Intel® Celeron®, Core (2) Duo®, Itanium®, Pentium®, Xeon®, and XScale® processors; and similar processors. Dual microprocessors, multi-core processors, and other multi-processor architectures may also be employed as the processing unit 702.

The system bus 706 provides an interface for system components including, but not limited to, the system memory 704 to the processing unit 702. The system bus 706 can be any of several types of bus structure that may further interconnect to a memory bus (with or without a memory controller), a peripheral bus, and a local bus using any of a variety of commercially available bus architectures. Interface adapters may connect to the system bus 706 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The computing architecture 700 may comprise or implement various articles of manufacture. An article of manufacture may comprise a computer-readable storage medium to store logic. Examples of a computer-readable storage medium may include any tangible media capable of storing electronic data, including volatile memory or non-volatile memory, removable or non-removable memory, erasable or non-erasable memory, writeable or re-writeable memory, and so forth. Examples of logic may include executable computer program instructions implemented using any suitable type of code, such as source code, compiled code, interpreted code, executable code, static code, dynamic code, object-oriented code, visual code, and the like. Embodiments may also be at least partly implemented as instructions contained in or on a non-transitory computer-readable medium, which may be read and executed by one or more processors to enable performance of the operations described herein.

The system memory 704 may include various types of computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (c.g., USB memory, solid state drives (SSD) and any other type of storage media suitable for storing information. In the illustrated embodiment shown in FIG. 7, the system memory 704 can include non-volatile memory 708 and/or volatile memory 710. A basic input/output system (BIOS) can be stored in the non-volatile memory 708.

The computing architecture 700 may include various types of computer-readable storage media in the form of one or more lower speed memory units, including an internal (or external) hard disk drive (HDD) 712, a magnetic floppy disk drive (FDD) 714 to read from or write to a removable magnetic disk 716, and an optical disk drive 718 to read from or write to a removable optical disk 720 (e.g., a CD-ROM or DVD). The HDD 712, FDD 714 and optical disk drive 720 can be connected to the system bus 706 by an HDD interface 722, an FDD interface 724 and an optical drive interface 726, respectively. The HDD interface 722 for external drive implementations can include at least one or both of Universal Serial Bus (USB) and IEEE 694 interface technologies.

The drives and associated computer-readable media provide volatile and/or nonvolatile storage of data, data structures, computer-executable instructions, and so forth. For example, a number of program modules can be stored in the drives and memory units 708, 712, including an operating system 728, one or more application programs 730, other program modules 732, and program data 734. In one embodiment, the one or more application programs 730, other program modules 732, and program data 734 can include, for example, the various applications and/or components of the messaging system 500.

A user can enter commands and information into the computer 701 through one or more wire/wireless input devices, for example, a keyboard 736 and a pointing device, such as a mouse 738. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices arc often connected to the processing unit 702 through an input device interface 740 that is coupled to the system bus 706, but can be connected by other interfaces such as a parallel port, IEEE 694 serial port, a game port, a USB port, an IR interface, and so forth.

A monitor 742 or other type of display device is also connected to the system bus 706 via an interface, such as a video adaptor 744. the monitor 742 may be internal or external to the computer 701. In addition to the monitor 742, a computer typically includes other peripheral output devices, such as speakers, printers, and so forth.

The computer 701 may operate in a networked environment using logical connections via wire and/or wireless communications to one or more remote computers, such as a remote computer 744. The remote computer 744 can be a workstation, a server computer, a router, a personal computer, portable computer, microprocessor-based entertainment appliance, a peer device or other common network node, and typically includes many or all of the elements described relative to the computer 701, although, for purposes of brevity, only a memory/storage device 746 is illustrated. The logical connections depicted include wire/wireless connectivity to a local area network (LAN) 748 and/or larger networks, for example, a wide area network (WAN) 750. Such LAN and WAN networking environments are commonplace in offices and companies, and facilitate enterprise-wide computer networks, such as intranets, all of which may connect to a global communications network, for example, the Internet.

When used in a LAN networking environment, the computer 701 is connected to the LAN 748 through a wire and/or wireless communication network interface or adaptor 752. the adaptor 752 can facilitate wire and/or wireless communications to the LAN 748, which may also include a wireless access point disposed thereon for communicating with the wireless functionality of the adaptor 752.

When used in a WAN networking environment, the computer 701 can include a modem 754, or is connected to a communications server on the WAN 750, or has other means for establishing communications over the WAN 750, such as by way of the Internet. The modem 754, which can be internal or external and a wire and/or wireless device, connects to the system bus 706 via the input device interface 740. In a networked environment, program modules depicted relative to the computer 701, or portions thereof, can be stored in the remote memory/storage device 746. It will be appreciated that the network connections shown arc exemplary and other means of establishing a communications link between the computers can be used.

The computer 701 is operable to communicate with wire and wireless devices or entities using the IEEE 802 family of standards, such as wireless devices operatively disposed in wireless communication (e.g., IEEE 802.13 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.13x (a, b, g, n, etc.) to provide secure, reliable, fast wireless connectivity. A Wi-Fi network can be used to connect computers to each other, to the Internet, and to wire networks (which use IEEE 802.3-related media and functions).

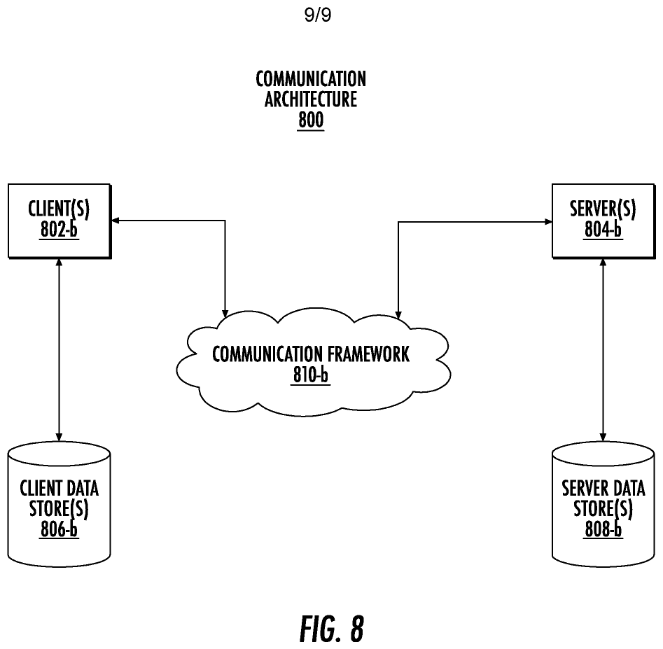

FIG. 8 is a block diagram depicting an exemplary communications architecture 800 suitable for implementing various embodiments as previously described. The communications architecture 800 includes various common communications elements, such as a transmitter, receiver, transceiver, radio, network interface, baseband processor, antenna, amplifiers, filters, power supplies, and so forth. The embodiments, however, are not limited to implementation by the communications architecture 800.

As shown in FIG. 8, the communications architecture 800 includes one or more clients 802 and servers 804. The clients 802 may implement the client device 510. The servers 804 may implement the server device 526. The clients 802 and the servers 804 are operatively connected to one or more respective client data stores 806 and server data stores 808 that can be employed to store information local to the respective clients 802 and servers 804, such as cookies and/or associated contextual information.

The clients 802 and the servers 804 may communicate information between each other using a communication framework 810. The communications framework 810 may implement any well-known communications techniques and protocols. The communications framework 810 may be implemented as a packet-switched network (e.g., public networks such as the Internet, private networks such as an enterprise intranet, and so forth), a circuit- switched network (e.g., the public switched telephone network), or a combination of a packet-switched network and a circuit-switched network (with suitable gateways and translators).

The communications framework 810 may implement various network interfaces arranged to accept, communicate, and connect to a communications network. A network interface may be regarded as a specialized form of an input output interface. Network interfaces may employ connection protocols including without limitation direct connect, Ethernet (e.g., thick, thin, twisted pair 10/100/1000 Base T, and the like), token ring, wireless network interfaces, cellular network interfaces, IEEE 802.8a-x network interfaces, IEEE 802.16 network interfaces, IEEE 802.20 network interfaces, and the like. Further, multiple network interfaces may be used to engage with various communications network types. For example, multiple network interfaces may be employed to allow for the communication over broadcast, multicast, and unicast networks. Should processing requirements dictate a greater amount speed and capacity, distributed network controller architectures may similarly be employed to pool, load balance, and otherwise increase the communicative bandwidth required by clients 802 and the servers 804. A communications network may be any one and the combination of wired and/or wireless networks including without limitation a direct interconnection, a secured custom connection, a private network (e.g., an enterprise intranet), a public network (e.g., the Internet), a Personal Area Network (PAN), a Local Area Network (LAN), a Metropolitan Area Network (MAN), an Operating Missions as Nodes on the Internet (OMNI), a Wide Area Network (WAN), a wireless network, a cellular network, and other communications networks.

The components and features of the devices described above may be implemented using any combination of discrete circuitry, application specific integrated circuits (ASICs), logic gates and/or single chip architectures. Further, the features of the devices may be implemented using microcontrollers, programmable logic arrays and/or microprocessors or any combination of the foregoing where suitably appropriate. It is noted that hardware, firmware and/or software elements may be collectively or individually referred to herein as “logic” or “circuit.”

It will be appreciated that the exemplary devices shown in the block diagrams described above may represent one functionally descriptive example of many potential implementations. Accordingly, division, omission or inclusion of block functions depicted in the accompanying figures does not infer that the hardware components, circuits, software and/or elements for implementing these functions would be necessarily be divided, omitted, or included in embodiments.

At least one computer-readable storage medium may include instructions that, when executed, cause a system to perform any of the computer-implemented methods described herein.

Some embodiments may be described using the expression “one embodiment” or “an embodiment” along with their derivatives. These terms mean that a particular feature, structure, or characteristic described in connection with the embodiment is included in at least one embodiment. The appearances of the phrase “in one embodiment” in various places in the specification are not necessarily all referring to the same embodiment. Moreover, unless otherwise noted the features described above are recognized to be usable together in any combination. Thus, any features discussed separately may be employed in combination with each other unless it is noted that the features are incompatible with each other.

With general reference to notations and nomenclature used herein, the detailed descriptions herein may be presented in terms of program procedures executed on a computer or network of computers. These procedural descriptions and representations are used by those skilled in the art to most effectively convey the substance of their work to others skilled in the art.

A procedure is here, and generally, conceived to be a self-consistent sequence of operations leading to a desired result. These operations are those requiring physical manipulations of physical quantities. Usually, though not necessarily, these quantities take the form of electrical, magnetic or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms arc to be associated with the appropriate physical quantities and arc merely convenient labels applied to those quantities.

Further, the manipulations performed arc often referred to in terms, such as adding or comparing, which arc commonly associated with mental operations performed by a human operator. No such capability of a human operator is necessary, or desirable in most cases, in any of the operations described herein, which form part of one or more embodiments. Rather, the operations are machine operations. Useful machines for performing operations of various embodiments include general purpose digital computers or similar devices.

Some embodiments may be described using the expression “coupled” and “connected” along with their derivatives. These terms are not necessarily intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Various embodiments also relate to apparatus or systems for performing these operations. This apparatus may be specially constructed for the required purpose or it may comprise a general purpose computer as selectively activated or reconfigured by a computer program stored in the computer. The procedures presented herein are not inherently related to a particular computer or other apparatus. Various general purpose machines may be used with programs written in accordance with the teachings herein, or it may prove convenient to construct more specialized apparatus to perform the required method steps. The required structure for a variety of these machines will appear from the description given.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than arc expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment. In the appended claims, the terms “including” and “in which” are used as the plain-English equivalents of the respective terms “comprising” and “wherein,” respectively. Moreover, the terms “first,” “second,” “third,” and so forth, are used merely as labels, and are not intended to impose numerical requirements on their objects.

What has been described above includes examples of the disclosed architecture. It is, of course, not possible to describe every conceivable combination of components and/or methodologies, but one of ordinary skill in the art may recognize that many further combinations and permutations are possible. Accordingly, the novel architecture is intended to embrace all such alterations, modifications and variations that fall within the spirit and scope of the appended claims.

1. A non-transitory computer-readable medium storing instructions that, when executed by one or more processors, cause the processors to:
interface with an analysis instrument configured to analyze a collection of samples, the samples comprising a test sample and a control sample;
receive results of an analysis of the collection of samples, the analysis comprising time-series measurements for the collection of samples;
align the time-series measurements of the collection of samples;
programmatically identify a sample of interest by comparing the aligned time-series measurements of the test sample and the control sample; and
generate an output comprising an identifier for the identified sample of interest.

2. The medium of claim 1, wherein the time-series measurements comprise one or more of spectral absorbance measurements, phosphorescence measurements, fluorescence measurements, or voltage measurements.

3. The medium of claim 1, wherein the time-series measurements comprise one or more of energy, force, torque, light, or position measurements, or the conversion of an energy, force, torque, light, or position measurement to an electrical signal.

4. The medium of claim 1, wherein:
the control sample does not include a component of interest and the test sample does include a component of interest;
aligning the time-series measurements comprises:
identifying one or more first peaks in the time-series measurement of the test sample,
identifying one or more second peaks in the time-series measurement of the control sample, and
allowing the first peaks and the second peaks to float relative to each other within a predefined tolerance window; and
programmatically identifying the sample of interest comprises subtracting or dividing a first time-series measurement of the control sample from a second time-series measurement of the test sample.

5. The medium of claim 1, wherein:
the samples comprise at least one of a protein, DNA, RNA, polysaccharide, lipid, polymer, or small molecules;
the sample of interest includes at least one of a protein, DNA, RNA, polysaccharide, lipid, polymer, or small molecules not present in the control sample; and
the analysis comprises an electrophoresis analysis.

6. The medium of claim 1, wherein programmatically identifying the sample of interest comprises:
computing a distance between the time-series measurement of the control sample and the time-series measurement of the test sample; and
selecting the control sample from among a plurality of control samples, the control sample selected as a match with the test sample based on identifying that the control sample has the smallest computed distance from the test sample from among to the plurality of control samples.

7. The medium of claim 1, wherein the output comprises a table of spectral peaks, and further storing instructions for identifying a database of experimental results and automatically submitting the output to the database.

8. A method comprising:
interfacing with an analysis instrument configured to analyze a collection of samples, the samples comprising a test sample and a control sample;
receiving results of an analysis of the collection of samples, the analysis comprising time- series measurements for the collection of samples;
aligning the time-series measurements of the collection of samples;
programmatically identifying a sample of interest by comparing the aligned time-series measurements of the test sample and the control sample; and
generating an output comprising an identifier for the identified sample of interest.

9. The method of claim 8, wherein the time-series measurements comprise one or more of spectral absorbance measurements, phosphorescence measurements, fluorescence measurements, or voltage measurements.

10. The method of claim 8, wherein the time-series measurements comprise one or more of energy, force, torque, light, or position measurements, or the conversion of an energy, force, torque, light, or position measurement to an electrical signal.

11. The method of claim 8, wherein:
the control sample does not include a component of interest and the test sample does include a component of interest;
aligning the time-series measurements comprises:
identifying one or more first peaks in the time-series measurement of the test sample,
identifying one or more second peaks in the time-series measurement of the control sample, and
allowing the first peaks and the second peaks to float relative to each other within a predefined tolerance window; and
programmatically identifying the sample of interest comprises subtracting or dividing a first time-series measurement of the control sample from a second time-series measurement of the test sample.

12. The method of claim 8, wherein:
the samples comprise at least one of a protein, DNA, RNA, polysaccharide, lipid, polymer, or small molecules;
the sample of interest includes at least one of a protein, DNA, RNA, polysaccharide, lipid, polymer, or small molecules not present in the control sample; and
the analysis comprises an electrophoresis analysis.

13. The method of claim 8, wherein programmatically identifying the sample of interest comprises:
computing a distance between the time-series measurement of the control sample and the time-series measurement of the test sample; and
selecting the control sample from among a plurality of control samples, the control sample selected as a match with the test sample based on identifying that the control sample has the smallest computed distance from the test sample from among to the plurality of control samples.

14. The method of claim 8, wherein the output comprises a table of spectral peaks, and further storing instructions for identifying a database of experimental results and automatically submitting the output to the database.

15. An apparatus comprising:
a hardware interface configured to communicate with an analysis instrument, the analysis instrument configured to analyze a collection of samples, the samples comprising a test sample and a control sample, wherein communicating with the analysis instrument comprises receiving results of an analysis of the collection of samples, the analysis comprising time-series measurements for the collection of samples;
a hardware processor configured to align the time-series measurements of the collection of samples, and to programmatically identify a sample of interest by comparing the aligned time-series measurements of the test sample and the control sample; and
a non-transitory computer-readable medium configured to store an output comprising an identifier for the identified sample of interest.

16. The apparatus of claim 15, wherein the time-series measurements comprise one or more of spectral absorbance measurements, phosphorescence measurements, fluorescence measurements, or voltage measurements.

17. The apparatus of claim 15, wherein the time-series measurements comprise one or more of energy, force, torque, light, or position measurements, or the conversion of an energy, force, torque, light, or position measurement to an electrical signal.

18. The apparatus of claim 15, wherein:
the control sample does not include a component of interest and the test sample does include a component of interest;
aligning the time-series measurements comprises:
identifying one or more first peaks in the time-series measurement of the test sample,
identifying one or more second peaks in the time-series measurement of the control sample, and
allowing the first peaks and the second peaks to float relative to each other within a predefined tolerance window; and
programmatically identifying the sample of interest comprises subtracting or dividing a first time-series measurement of the control sample from a second time-series measurement of the test sample.

19. The apparatus of claim 15, wherein:
the samples comprise at least one of a protein, DNA, RNA, polysaccharide, lipid, polymer, or small molecules;
the sample of interest includes at least one of a protein, DNA, RNA, polysaccharide, lipid, polymer, or small molecules not present in the control sample; and
the analysis comprises an electrophoresis analysis.

20. The apparatus of claim 15, wherein programmatically identifying the sample of interest comprises:
computing a distance between the time-series measurement of the control sample and the time-series measurement of the test sample; and
selecting the control sample from among a plurality of control samples, the control sample selected as a match with the test sample based on identifying that the control sample has the smallest computed distance from the test sample from among to the plurality of control samples.
